(19) United States
(12) Patent Application Publication
(10) Pub. No.: US 2021/0134443 A1
(43) Pub. Date: May 6, 2021

(54) Correlating Patient Health Characteristics with Relevant Treating Clinicians

(71) Applicant: Tridiuum, Inc., Philadelphia, PA (US)

(72) Inventors: Mark Redlus, Westampton, NJ (US); Gabriel Eichler, Belmont, MA (US); Sudhir Varma, Princeton, NJ (US); Laura Dietzen, Glen Carbon, IL (US); Tina Harralson, Jensen Beach, FL (US); James Flaherty, Philadelphia, PA (US); Deborah Cypher, Elkton, FL (US); Scott Johnson, Scottsdale, AZ (US)

(21) Appl. No.: 16/807,085

(22) Filed: Mar. 2, 2020

Related U.S. Application Data
(60) Provisional application No. 62/928,317, filed on Oct. 30, 2019.

(51) Int. Cl. G16H 40/20; G16H 50/70; G16H 10/60; G16H 50/50; G16H 50/20; G16H 50/30; G16H 70/60; A61B 5/16

(52) U.S. Cl. CPC G16H 40/20; G16H 50/70; G16H 10/60; A61B 5/165; G16H 50/20; G16H 50/30; G16H 70/60; G16H 50/50

(57) ABSTRACT

A method builds models for matching patients to clinicians. For each of a plurality of patients, the method retrieves a plurality of clinician selection characteristics and a temporal sequence of two or more health assessments. Each health assessment tracks a plurality of health status conditions and a treating clinician. The method then forms a respective feature vector that includes the clinician selection characteristics, indicators for health characteristics, a computed health status change according to the temporal sequence of two or more health assessments, and an identifier of the treating clinician. The method uses the feature vectors to train a model that correlates sets of clinician selection characteristics and health characteristics to optimal treating clinicians. The method then stores the trained model in a database for subsequent use in matching new patients to treating clinicians.

### RELATED APPLICATIONS

This application claims priority to U.S. Provisional Patent Application Ser. No. 62/928,317, filed Oct. 30, 2019, which is incorporated by reference herein in its entirety.

This application is related to U.S. patent application Ser. No. ______, filed Mar. 2, 2020, entitled “Correlating Patient Health Characteristics with Relevant Treating Clinicians” (Attorney Docket Number 120396-5002-US), which is incorporated by reference herein in its entirety.

### TECHNICAL FIELD

The disclosed implementations relate generally to providing health care referrals and more specifically to systems and methods for tracking patients' progress and providing clinician referrals to patients.

### BACKGROUND

Health care referrals are largely fulfilled through professional networks, assigned based on availability, and/or proximity to patients. In some cases, the clinicians are evaluated and ranked, either through peer review or patient feedback.

### SUMMARY

The existing processes for healthcare referrals have little information to determine the likelihood of compatibility between clinicians and patients. This poses a problem for developing strong patient-clinician relationships, resulting in poor patient retention and a failure to provide the patient with the best healthcare possible. These effects can lead to ineffective referrals, may be detrimental to the health and well-being of the patient, and may lead to delayed or stunted progress in the patient's condition. Current methods fail to provide patients with referrals to clinicians that have a high likelihood of compatibility and treatment success for the patient.

To provide effective healthcare referrals with a high chance of success, it is important to track patient information and treatment progress in order to understand factors that lead to successful patient-clinician relationships and accurately predict the likelihood of success between potential patients and clinicians. Existing techniques do not track patient progress and do not use such information to identify future patient-clinician pairings that will have the high chance of success.

Accordingly, there is a need for tools that can accurately calculate and predict the likelihood of compatibility and treatment success when making healthcare referrals for patients. There is also a need for tools that employ such calculations and predictions to allow systems to effectively guide or assist healthcare referrals. One solution to the problem is to monitor patients' progress for each patient-clinician pair that are already working together. By tracking the patients' progress, factors that predict compatibility and good therapeutic fit can be identified and leveraged to improve future healthcare referrals. This technique allows referrals to be made on a personalized basis (e.g., based on the personalized needs of a specific patient and on a clinician's history of success with patients who have that specific need), rather than on a global basis (e.g., referring a patient to the top ranked therapist in the area). For each patient, clinicians are being evaluated relative to the personalized needs and characteristics of the patient, instead of being evaluated globally based on overall skill, performance, or experience. This technique produces (e.g., generates, provides) referrals based on predicted fit to personal characteristics of each patient, thereby improving the quality of referrals, increasing patient retention rates, and increasing success rate or improvement rate of the patient's condition.

Additionally, a scheduling and triage process of the technique can lead to improved service to new patients. For example, in an initial testing phase that included over 500 new patients, nearly 7% were identified as being at risk of being a danger to themselves or others, and each of the identified patients were able to be treated by a clinician immediately (e.g., within the same day, within a 2 hour window, or within a 4 hour window). During the initial testing phase, the amount of time between a referral request and the patient being able to be treated by a matched clinician was decreased from a 28-day process down to less than 7 days, more than a 75% reduction in time.

In accordance with some implementations, a method for building a model for matching patients to clinicians executes at an electronic device with a display, one or more processors, and memory. For example, the electronic device can be a smart phone, a tablet, a notebook computer, a desktop computer, an individual server computer, or a server system (e.g., running in the cloud). For each of a plurality of patients, the device retrieves a respective plurality of clinician selection characteristics and a respective temporal sequence of two or more health assessments. Each health assessment tracks a plurality of health status conditions and a respective treating clinician. For each of the patients, the device forms a respective feature vector that includes the respective clinician selection characteristics, indicators for a plurality of health characteristics determined from the health status conditions, a computed health status change according to the temporal sequence of two or more health assessments, and an identifier of the respective treating clinician. The device then uses the feature vectors to train a model that correlates sets of clinician selection characteristics and health characteristics to optimal treating clinicians. The device then stores the trained model in a database for subsequent use in matching new patients to treating clinicians.

In some implementations, the plurality of patients are mental health patients, the plurality of health status conditions are mental health status conditions, and the health assessments are behavioral health assessments.

In some implementations, each feature vector further includes one or more physical health characteristics measured by tests other than the health assessments.

In some implementations, each health assessment corresponds to a respective patient-clinician visit.

In some implementations, for each of the health assessments, the device computes a composite health score. Each health status change is computed as a difference between composite health scores.

In some implementations, for each health assessment, the device computes a composite health score. The health status change for each patient is computed based on two or more of the composite health scores for a respective patient.

In some implementations, the device tests the trained model by comparing results of the trained model to at least a component of the composite score.

In some implementations, the device also compares the health status change for the respective patient to an expected treatment response trend.

In some implementations, the expected treatment response trend is calculated using hierarchical linear modeling based on normative data for patients with one or more similar clinician selection characteristics to the respective patient

In some implementations, the device determines whether the health status change of the respective patient is statistically significant.

In some implementations, the health assessments further tracks one or more characteristics of interactions between patients and treating clinicians.

In some implementations, the one or more characteristics measure symptoms known to be correlated with a set of preselected medical conditions.

In some implementations, the device tests the trained model by comparing results of the trained model to one or more of: emergency room utilization records, hospital admissions records, and medical comorbidity code records.

In accordance with some implementations, a method of matching patients to clinicians executes at an electronic device with a display, one or more processors, and memory. For example, the electronic device can be a smart phone, a tablet, a notebook computer, a desktop computer, a server computer, a system of server computers, or a wearable device such as a smart watch. The device receives a health assessment from a user (e.g., the user completes/fills out/provides information for the health assessment via the device). The health assessment includes a plurality of clinician selection characteristics and a plurality of health status conditions. The device then retrieves a trained model. The model was trained according to a plurality of patients. Each patient provided a respective temporal sequence of health assessments during treatment by a respective treating clinician. The device now forms a feature vector, which includes the plurality of clinician selection characteristics and a plurality of health characteristics determined from the health status conditions. The device applies the trained model to the feature vector to generate a list of candidate treating clinicians who have optimally treated patients whose clinician selection characteristics and determined health characteristics correlate with the health assessment from the user. The device then provides the generated list of candidate treating clinicians to the user for selection.

In some implementations, the feature vector includes one or more user preferences that specify clinician selection characteristics for treating clinicians. Applying the trained model includes using the specified clinician selection characteristics for treating clinicians to generate the list of candidate treating clinicians.

In some implementations, the device receives user specification of one or more user preferences for clinician selection characteristics for treating clinicians and filters the generated list of candidate treating clinicians according to the user preferences for clinician selection characteristics for treating clinicians.

In some implementations, a first user preference for clinician selection characteristics for treating clinicians is a gender identifier. Filtering the generated list of candidate treating clinicians includes comparing the gender identifier to gender identifiers of candidate treating clinicians included in the generated list.

In some implementations, the feature vector further includes an identifier of urgency and/or an identifier of illness severity.

In some implementations, the device receives user specification of a preferred health care approach and the feature vector includes both the preferred health care approach and a suitability score for the preferred health care approach.

In some implementations, the device receives user specification of a user location and filters the generated list of candidate treating clinicians by comparing the user location to locations of candidate treating clinicians on the generated list.

In some implementations, the device receives a scheduling preference of the user, compares the scheduling preference of the user to availability of candidate treating clinicians on the generated list, and updates the list of candidate treating clinicians to exclude treating clinicians who do not have at least one availability that matches with the scheduling preference of the user.

Typically, an electronic device includes one or more processors, memory, a display, and one or more programs stored in the memory. The programs are configured for execution by the one or more processors and are configured to perform any of the methods described herein.

In some implementations, a non-transitory computer readable storage medium stores one or more programs configured for execution by a computing device having one or more processors, memory, and a display. The one or more programs are configured to perform any of the methods described herein.

Thus methods and systems are disclosed that correlate patient health characteristics with relevant treating clinicians.

Both the foregoing general description and the following detailed description are exemplary and explanatory, and are intended to provide further explanation of the invention as claimed.

### BRIEF DESCRIPTION OF THE DRAWINGS

For a better understanding of these systems, methods, and graphical user interfaces, as well as additional systems, methods, and graphical user interfaces that correlate patients with treating clinicians, refer to the Description of Implementations below, in conjunction with the following drawings, in which like reference numerals refer to corresponding parts throughout the figures.

FIG. 1A illustrates training a therapeutic referral model in accordance with some implementations.

FIG. 1B illustrates using a therapeutic referral model in accordance with some implementations.

FIG. 2A is a block diagram illustrating a computing device according to some implementations.

FIG. 2B is a block diagram illustrating a server according to some implementations.

FIG. 3A illustrates how a therapeutic referral model is trained according to some implementations.

FIG. 3B provides an example of a health assessment according to some implementations.

FIG. 3C provides an example of a health progress according to some implementations.

FIG. 3D illustrates a box plot showing the effects of providing personalized referrals according to some implementations.

FIG. 4A illustrates using a therapeutic referral model for providing healthcare referrals according to some implementations.

FIG. 4B is an example of clinician data according to some implementations.

FIGS. 5A and 5B provide a flow diagram of a method for building a model for matching patients to clinicians according to some implementations.

FIGS. 6A and 6B provide a flow diagram of a method for matching patients to clinicians according to some implementations.

Reference will now be made to implementations, examples of which are illustrated in the accompanying drawings. In the following description, numerous specific details are set forth in order to provide a thorough understanding of the present invention. However, it will be apparent to one of ordinary skill in the art that the present invention may be practiced without requiring these specific details.

### DESCRIPTION OF IMPLEMENTATIONS

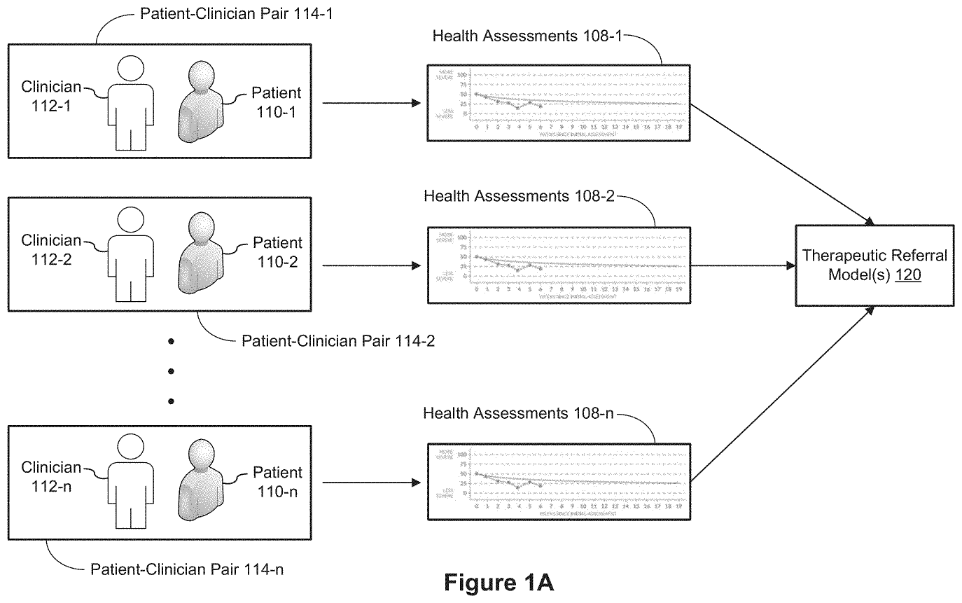

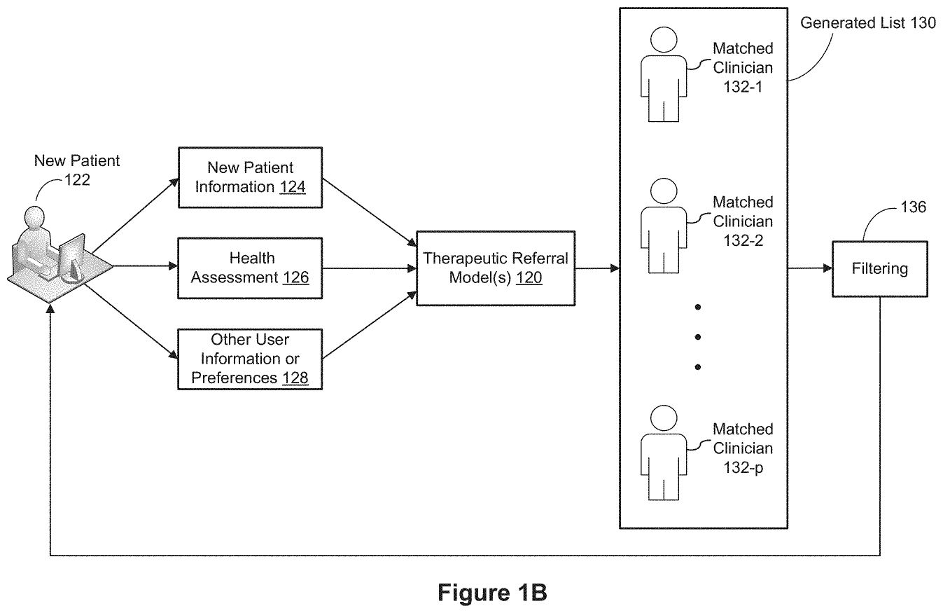

FIG. 1A illustrates generating therapeutic referral model(s) 120 using information from clinician-patient pairs. For example, for a first patient-clinician pair 114-1, which includes a first patient 110-1 and a first clinician 112-1, a health assessment 108 (e.g., a behavioral health assessment, a mental health assessment, a physical health assessment, or a pre/post-surgical health assessment) is recorded at the end of each treatment session (e.g., therapeutic session, counseling appointment, or medical appointment). The health assessment may include information compiled by a post-session questionnaire completed by the patient 110-1 and/or the clinician 112-1. The health assessment includes one or more health status conditions of the patient 110-1. Some examples of information that may be included in the health assessment are gender, current weight, current height, current condition (e.g., current state of mind, current pain level), a patient self-reported level of progress, current medications, adjustments to medications (if any), and a patient's evaluation of the treatment session (e.g., whether the patient felt his/her concerns were addressed, how much benefit does the patient think that he/she received from the treatment session). The health assessment may also include feedback from the clinician 112-1 on the patient's progress and/or the clinician's evaluation of the treatment session (e.g., how did the patient 110-1 respond to the treatment, a comparison of the patient at the beginning versus the end of the treatment session, and/or whether the clinician thinks that the techniques employed during the treatment session were beneficial and/or effective in helping the patient 110-1). When a temporal sequence of at least two health assessments are received for a same clinician-patient pair 114, a set of health assessments 108 is formed. For example, for the first patient-clinician pair 114-1, a first set of health assessments 108-1 is received. In the case where the first patient-clinician pair 114-1 have had two treatment sessions, the set of health assessments 108-1 includes three health assessments: an intake health assessment corresponding to the patient at intake (e.g., before treatment), a health assessment corresponding to the first treatment session and a health assessment corresponding to a second treatment session. A second set of health assessments 108-2 is received from a second patient-clinician pair that includes a second patient 110-2 and a second clinician 112-2. In some cases, the second clinician 112-2 may be different from the first clinician 112-1. Alternatively, since one clinician may treat many different patients, the second clinician 112-2 may be the same as the first clinician 112-1. This process is repeated for n patient-clinician pairs so that n sets of health assessments are received.

The information received from the sets of health assessments 108 are then broken down into indicators for health characteristics. Some examples of indicators for health characteristics include diagnosis, prognosis, medical history, age, pre-existing conditions, and gender.

Additionally, the set of health assessments 108 is used to calculate a health status change for a respective patient 110 of the patient-clinician pair 114 corresponding to the set of health assessments 118. For example, a health status change for a respective patient 110 may be the difference between a patient's health scores from the two most recent health assessments received from the patient.

In some implementations, in addition to the health assessments, a plurality of clinician selection characteristics may be recorded for each clinician 112. The clinician selection characteristics may include information regarding the clinician 112, such as the clinician's gender, age, office location, specialty field(s), expertise or certification in specific techniques or methods, level of experience (e.g., number of years of experience), certifications, or affiliations.

A feature vector is formed using the indicators for health characteristics, the clinician selection characteristics, the health status change, and an identifier of the treating clinician. The feature vector is then used to train one or more therapeutic referral model(s) 120 so that the therapeutic referral model(s) 120 can correlate sets of clinician selection characteristics and health characteristics to optimal treating clinicians, thereby matching patients with the clinicians that are predicted to have good success rates in treating the patient.

The feature vectors incorporate patient-specific and clinician-specific factors. Examples of patient-specific factors include age, gender, as well as other information retrieved from a health assessment, such as a measure of treatability, medical history (e.g., pre-existing conditions, and/or family history), and diagnosed diseases (e.g., diagnosed with depression, cancer, a broken leg). Examples of clinician-specific factors include field of expertise, certifications or trainings, clinical degree(s), years of experience, and the clinician's contractual relationship to care system (e.g., in-network provider versus out-of-network provider). In some implementations, the feature vectors includes joint-factors that characterize the relationship between the patient and the clinician, including health status change for the patient while being treated by a specific clinician. Examples of joint-factors include patient-clinician combined factors, a measure of therapeutic alliance, and productivity of therapy. The therapeutic referral model(s) 120 are trained to account for patient-specific factors in order to provide an accurate estimate or assessment of the patient-clinician factors during the training process.

FIG. 1B illustrates using one or more therapeutic referral model(s) 120 to match a patient with a clinician. When a new patient 122 needs a referral to a clinician, the new patient 122 provides the therapeutic referral model(s) 120 with information regarding the patient (e.g., new patient information 124) and a health assessment 126 (e.g., a behavioral health assessment, a mental health assessment, a physical health assessment, and/or a pre/post-surgical health assessment). The new patient 122 may be a patient that is not currently part of the system (e.g., a new member), or may be a current patient (e.g., an existing member) in the system who needs a new referral. The new patient information 124 may include information such as the patient's name, address, insurance information (e.g., insurance coverage, in-network providers), gender, age, disease, and/or medical concerns. The health assessment 126 may include the same information as the health assessments described above. In some cases, the health assessment 126 may include some information or may have one or more missing fields compared to the health assessments described above. For example, the health assessment 126 may not have information regarding a clinician's assessment of the patient's progress or condition. In another example, the health assessment 126 may not have information regarding the patient's evaluation of a treatment session.

In some cases, the new patient 122 also provides other user information or preferences 128 to the therapeutic referral model(s) 120. The other user information or preferences 128 may include information such as the patient's preference in clinician gender, location, or the patient's preferred appointment times.

In some implementations, the therapeutic referral models 120 provide a score for each candidate clinician for treating the new patient 122. The scores are computed based on patient-specific factors and patient-clinician factors for each patient-clinician type. In some implementations, the scores are a summation of patient-specific factors and patient-clinician factors for each patient-clinician type. In some implementations, the scores are computed using machine learning, such as a neural network or a random forest of decision trees. The clinicians are ranked based on their scores. The therapeutic referral model(s) 120 then provide a generated list 130 of one or more matched clinicians 132 (e.g., matched clinicians 132-1, 132-2, . . . , 132-p). In some implementations, the generated list 130 of matched clinician(s) 132 are then filtered 136 based on the preferences 128 provided by the new patient 122. The generated list 130 of matched clinicians 132 is then provided to the new patient 122.

Thus, the therapeutic referral model(s) 120 provide a new patient 122 with a personalized referral that is based on information or characteristics of the new patient 122. The new patient 122 is not simply referred to the “best” clinician in the area or field, but rather, the new patient is matched with a clinician that is a best fit to the new patient's needs based on personal information provided by the new patient 122.

Initial testing suggests that patients who see a clinician that is matched in the patient's top decile (e.g., 90% percentile or greater) achieve medical outcomes (e.g., behavioral or mental health outcomes) that are approximately 2 times better compared to patients who receive care from clinicians who are not as well matched to the patients.

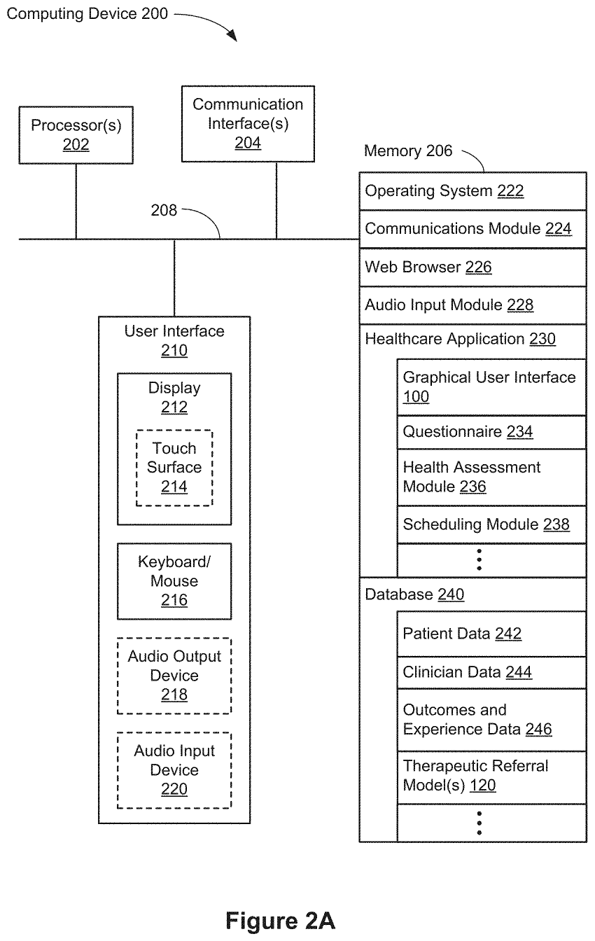

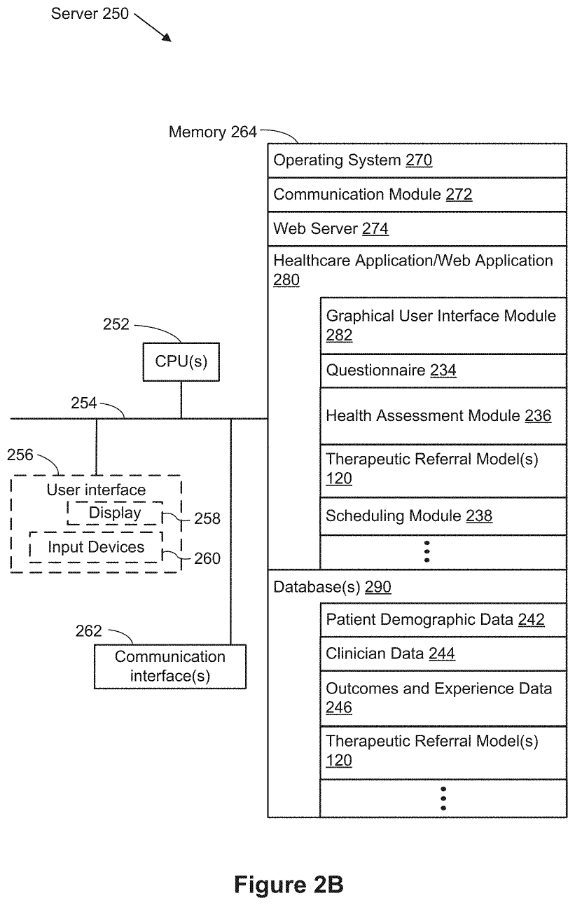

FIG. 2A is a block diagram illustrating a computing device 200, corresponding to a computing system, which can train and/or execute therapeutic referral model(s) 120 in accordance with some implementations. Various examples of the computing device 200 include a desktop computer, a laptop computer, a tablet computer, a server computer, a server system, a wearable device such as a smart watch, and other computing devices that have a processor capable of training and/or running therapeutic referral model(s) 120. The computing device 200 may be a data server that hosts one or more databases (e.g., database of images or videos), models, or modules, or may provide various executable applications or modules. The computing device 200 typically includes one or more processing units (processors or cores) 202, one or more network or other communications interfaces 204, memory 206, and one or more communication buses 208 for interconnecting these components. The communication buses 208 optionally include circuitry (sometimes called a chipset) that interconnects and controls communications between system components. The computing device 200 typically includes a user interface 210. The user interface 210 typically includes a display device 212 (e.g., a screen or monitor). In some implementations, the computing device 200 includes input devices such as a keyboard, mouse, and/or other input buttons 216. Alternatively or in addition, in some implementations, the display device 212 includes a touch-sensitive surface 214, in which case the display device 212 is a touch-sensitive display. In some implementations, the touch-sensitive surface 214 is configured to detect various swipe gestures (e.g., continuous gestures in vertical and/or horizontal directions) and/or other gestures (e.g., single/double tap). In computing devices that have a touch-sensitive display 214, a physical keyboard is optional (e.g., a soft keyboard may be displayed when keyboard entry is needed). The user interface 210 also includes an audio output device 218, such as speakers or an audio output connection connected to speakers, earphones, or headphones. Furthermore, some computing devices 200 use a microphone 220 and voice recognition software to supplement or replace the keyboard. An audio input device 220 (e.g., a microphone) captures audio (e.g., speech from a user).

The memory 206 includes high-speed random-access memory, such as DRAM, SRAM, DDR RAM, or other random-access solid-state memory devices; and may include non-volatile memory, such as one or more magnetic disk storage devices, optical disk storage devices, flash memory devices, or other non-volatile solid-state storage devices. In some implementations, the memory 206 includes one or more storage devices remotely located from the processors 202. The memory 206, or alternatively the non-volatile memory devices within the memory 206, includes a non-transitory computer-readable storage medium. In some implementations, the memory 206 or the computer-readable storage medium of the memory 206 stores the following programs, modules, and data structures, or a subset or superset thereof:
an operating system 222, which includes procedures for handling various basic system services and for performing hardware dependent tasks;
a communications module 224, which is used for connecting the computing device 200 to other computers and devices via the one or more communication network interfaces 204 (wired or wireless), such as the Internet, other wide area networks, local area networks, metropolitan area networks, and so on;
a web browser 226 (or other application capable of displaying web pages), which enables a user to communicate over a network with remote computers or devices;
an audio input module 228 (e.g., a microphone module) for processing audio captured by the audio input device 220. The captured audio may be sent to a remote server and/or processed by an application executing on the computing device 200 (e.g., health care application 230);
a health care application 230, which includes a graphical user interface 100 that allows a user to navigate the health care application 230, such as accessing and filling out a questionnaire 234, or viewing one or more health assessments via a health assessment module 236. For example, a new patient 122 may use the graphical user interface 100 of the health care application 230 to fill out a questionnaire 234 that corresponds to any of the new patient's information 124, the new patient's health assessment 126, and other user information or preferences 128. In another example, an existing patient (e.g., the patient 110-1 in FIG. 1A) may use the graphical user interface 100 of the health care application 230 to fill out a questionnaire 234 following a treatment session. The information input by users is then compiled by the health assessment module 236 in order to create sets of health assessments for each patient-clinician pair 114. The health care application 230 may also utilize the therapeutic referral model(s) 120 to match a new patient 122 with one or more clinicians. The therapeutic referral model(s) 120 take patient-clinician alliance and therapeutic fit into account when generating the list 130 of matched clinicians 132. In some implementations, the health care application 230 may utilize a scheduling module 238 to filter or exclude matched clinicians 132 who do not have at least some availability that matches the patient's scheduling preference. In some implementations, the health care application 230 utilizes the scheduling module 238 to schedule an appointment for the patient with a selected clinician;
a database 240, which stores information, such as patient data 242, clinician data 244, Outcomes and Experiences Data 246, and one or more therapeutic referral module(s) 120. Patient data 242 may include demographic information about each patient such as age and gender, as well as each patient's medical information such as pre-existing conditions, diagnosis, insurance coverage, previous treatment methods, previous treating clinicians, and current treating clinician. Additionally, patient data 242 may include other patient information or preferences 128 as described above. In some implementations, the patient information includes social determinants, such as homelessness. Clinician data 244 may include clinician selection characteristics, which may correspond to one or more user preferences 128. For example, clinician selection characteristics for a respective clinician may include the clinician's gender, field of expertise, schedule, office location, level of experience, certifications, and accreditations. Outcomes and Experiences 246 may include notes or values that are retrieved from questionnaires 234 or health assessments. For example, information stored as part of Outcomes and Experiences 246 may include health scores or composite health scores retrieved from health assessment, health status change scores that are computed (e.g., calculated) from two or more sequential (e.g., temporal) health assessments for the same patient-clinician pair 114.

In some implementations, the memory 206 stores metrics and/or scores determined by the therapeutic referral model(s) 120. In addition, the memory 206 may store thresholds and other criteria, which are compared against the metrics and/or scores determined by the health assessment module 236. For example, the health assessment module 236 may determine (e.g., calculate) a confidence level or an accuracy score for each health score or health status change score.

Each of the above identified executable modules, applications, or sets of procedures may be stored in one or more of the previously mentioned memory devices, and corresponds to a set of instructions for performing a function described above. The above identified modules or programs (i.e., sets of instructions) need not be implemented as separate software programs, procedures, or modules, and thus various subsets of these modules may be combined or otherwise re-arranged in various implementations. In some implementations, the memory 206 stores a subset of the modules and data structures identified above. Furthermore, the memory 206 may store additional modules or data structures not described above.

Although FIG. 2A shows a computing device 200, FIG. 2A is intended more as a functional description of the various features that may be present rather than as a structural schematic of the implementations described herein. In practice, and as recognized by those of ordinary skill in the art, items shown separately could be combined and some items could be separated.

FIG. 2B is a block diagram of a server 250 in accordance with some implementations. A server 250 may host one or more databases 240 or may provide various executable applications or modules. A server 250 typically includes one or more processing units/cores (CPUs) 252, one or more network interfaces 262, memory 264, and one or more communication buses 254 for interconnecting these components. In some implementations, the server 240 includes a user interface 256, which includes a display 258 and one or more input devices 260, such as a keyboard and a mouse. In some implementations, the communication buses 254 include circuitry (sometimes called a chipset) that interconnects and controls communications between system components.

In some implementations, the memory 264 includes high-speed random-access memory, such as DRAM, SRAM, DDR RAM, or other random-access solid-state memory devices, and may include non-volatile memory, such as one or more magnetic disk storage devices, optical disk storage devices, flash memory devices, or other non-volatile solid-state storage devices. In some implementations, the memory 264 includes one or more storage devices remotely located from the CPU(s) 252. The memory 264, or alternatively the non-volatile memory devices within the memory 264, comprises a non-transitory computer readable storage medium.

In some implementations, the memory 264, or the computer readable storage medium of the memory 264, stores the following programs, modules, and data structures, or a subset thereof:
an operating system 270, which includes procedures for handling various basic system services and for performing hardware dependent tasks;
a network communication module 272, which is used for connecting the server 250 to other computers via the one or more communication network interfaces (wired or wireless) and one or more communication networks, such as the Internet, other wide area networks, local area networks, metropolitan area networks, and so on;
a web server 274 (such as an HTTP server), which receives web requests from users and responds by providing responsive web pages or other resources;
a health care application or a health care web application 280, which may be downloaded and executed by a web browser 226 on a user's computing device 200. In general, a health care web application 280 has the same functionality as a desktop health care application 230, but provides the flexibility of access from any device at any location with network connectivity, and does not require installation and maintenance. In some implementations, the health care web application 280 includes various software modules to perform certain tasks. In some implementations, the health care web application 280 includes a graphical user interface module 282, which provides the user interface for all aspects of the health care web application 280;
in some implementations, the health care web application 280 includes questionnaires 234, a health assessment module 236, therapeutic referral models 120, and a scheduling module 238, as described above for a computing device 200;
one or more databases 290, which store data used or created by the health care web application 280 or health care application 230. The databases 290 may store patient data 242, clinician data 244, Outcomes and Experiences Data 246, and one or more therapeutic referral module(s) 120 as described above.

Each of the above identified executable modules, applications, or sets of procedures may be stored in one or more of the previously mentioned memory devices, and corresponds to a set of instructions for performing a function described above. The above identified modules or programs (i.e., sets of instructions) need not be implemented as separate software programs, procedures, or modules, and thus various subsets of these modules may be combined or otherwise re-arranged in various implementations. In some implementations, the memory 264 stores a subset of the modules and data structures identified above. In some implementations, the memory 264 stores additional modules or data structures not described above.

Although FIG. 2B shows a server 250, FIG. 2B is intended more as a functional description of the various features that may be present rather than as a structural schematic of the implementations described herein. In practice, and as recognized by those of ordinary skill in the art, items shown separately could be combined and some items could be separated. In addition, some of the programs, functions, procedures, or data shown above with respect to a server 250 may be stored or executed on a computing device 200. In some implementations, the functionality and/or data may be allocated between a computing device 200 and one or more servers 250. Furthermore, one of skill in the art recognizes that FIG. 2B need not represent a single physical device. In some implementations, the server functionality is allocated across multiple physical devices that comprise a server system. As used herein, references to a “server” include various groups, collections, or arrays of servers that provide the described functionality, and the physical servers need not be physically collocated (e.g., the individual physical devices could be spread throughout the United States or throughout the world).

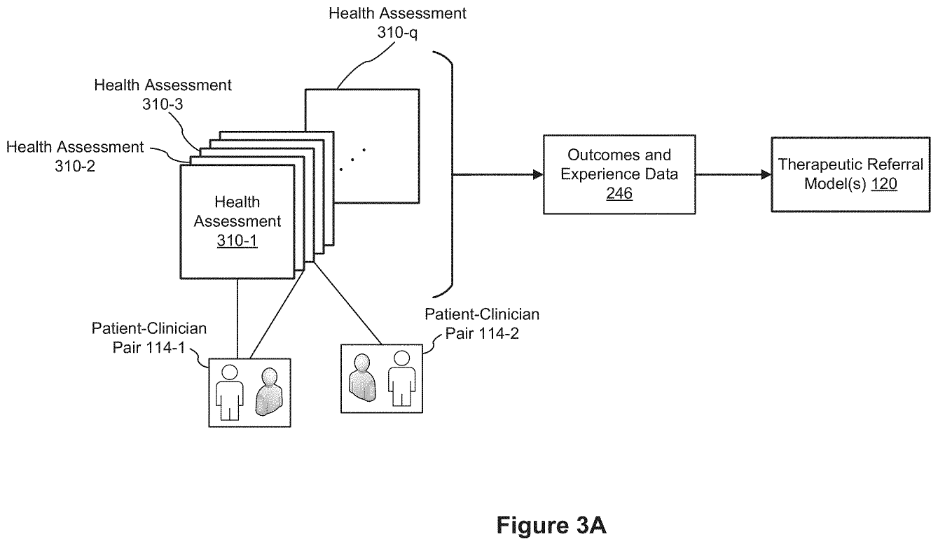

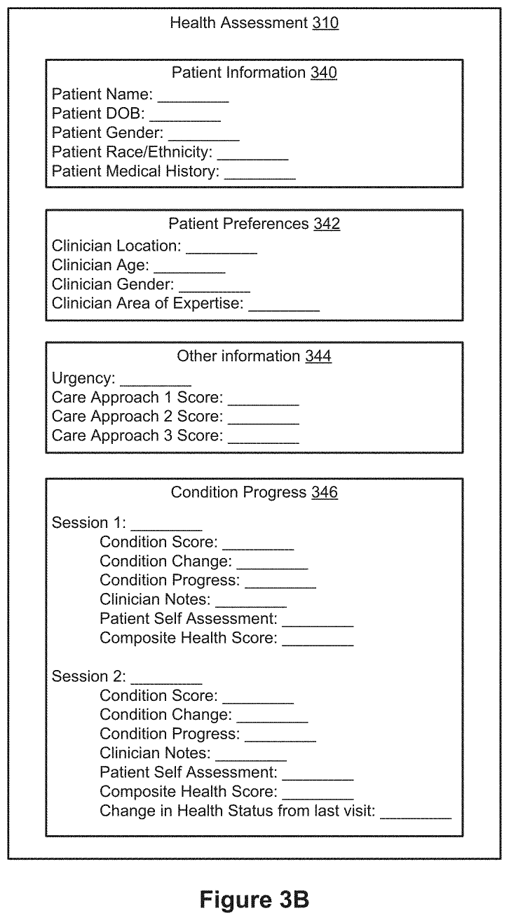

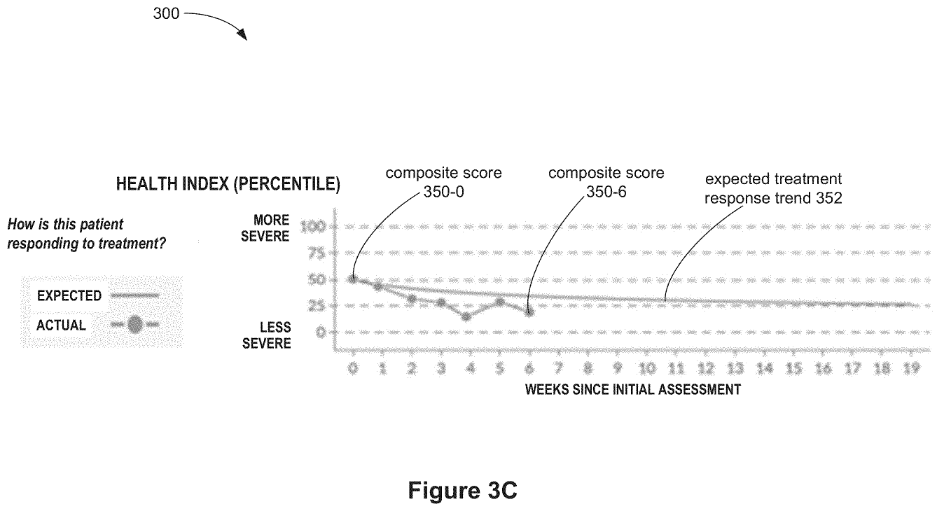

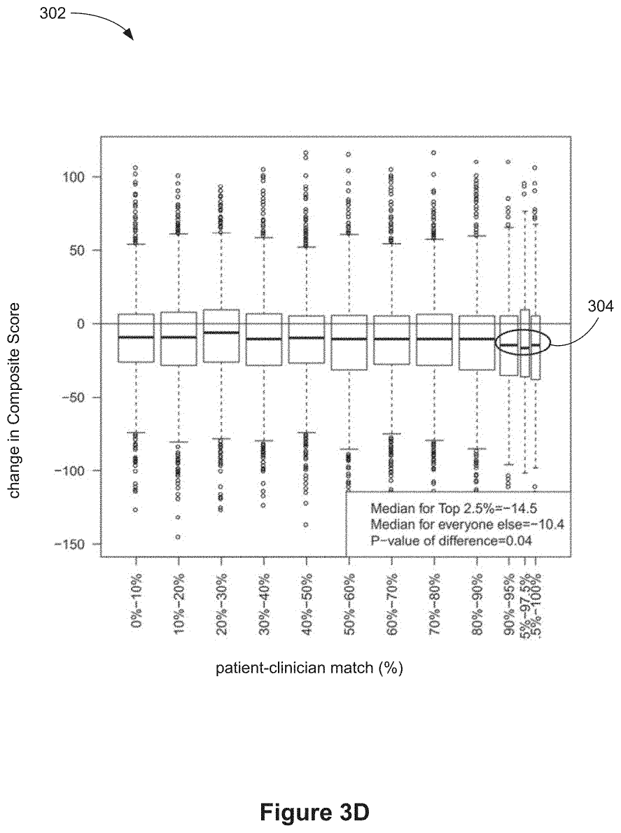

FIG. 3A illustrates how therapeutic referral model(s) 120 are trained according to some implementations. In order to train the therapeutic referral model(s) 120, the health assessment module 236 receives a plurality of health assessments 310 (e.g., behavioral health assessments, mental health assessments, physical health assessments, and/or pre/post-surgical health assessments). Each health assessment 310 corresponds to a respective patient clinician pair 114 and a respective treatment session. For example, a first health assessment 310-1 may correspond to a first patient-clinician pair 114-1 after a first treatment session. In some implementations, where the patient has seen the same clinician more than once, one or more other health assessments 310 (e.g., a second health assessment 310-2) may also correspond to the first patient-clinician pair 114-1 (e.g., the second health assessment 310-2 may correspond to the first patient-clinician pair 114-1 after a second treatment session, and so on and so forth). Thus, while q number of health assessments 310 may be collected, the number of patient-clinician pairs 114 may be equal to or less than q since one or more health assessments 310 may correspond to the same patient-clinician pair 114. Additionally, in some implementations, such as when a patient has seen multiple clinicians, two different patient-clinician pairs 114 may have a same patient but a different clinician. For example, a second patient-clinician pair 114-2, different from the first patient-clinician pair 114-1 may have the same patient but a different clinician. Similarly, a clinician may treat more than one patient and thus one clinician may be included in more than one patient-clinician pair 114.

The health assessment module 236 extracts information from each health assessment 310 and forms the Outcomes and Experience Data 246. For example, for each health assessment 310, the health assessment module 236 may compute a health status condition score and/or for each patient-clinician pair 114, and the health assessment module 236 may compute a health status condition change. The health status condition score and the health status condition change, along with any notes provided by a patient or clinician may be stored as part of the Outcomes and Experience Data 246. The Outcomes and Experience Data 246 is then used to train one or more therapeutic referral model(s) 120 so that the therapeutic referral model(s) 120 can learn and make connections as to what characteristics lead to a good patient-clinician alliance and good therapeutic fit. As training progresses, the therapeutic referral model(s) 120 are able to identify a combination of health assessment variables and associated weights that most approximates the benefit that a respective clinician provides to each patient. The therapeutic referral model(s) 120 are also able to identify particular practice patterns of clinicians that are associated with the respective clinician's success in treating patients with different medical issues (e.g., different behavioral or mental issues) and needs.

In a first example, the plurality of health assessments 310 may be behavioral health assessments that include information regarding the mental health or behavioral health of a respective patient. Information from the behavioral health assessments 310 is then stored in Outcomes and Experiences data 246 and used to train one or more therapeutic referral model(s) 120 to match new patients to therapists that are predicted to have a high alliance and/or therapeutic fit with the new patients.

In another example, the plurality of health assessments 310 may be physical health assessments that include information regarding the physical health of a respective patient, such as range of motion for a patient who is recovering from knee surgery. Information from the physical health assessments 310 is then stored in Outcomes and Experiences data 246 and used to train one or more therapeutic referral model(s) 120 to match new patients to physical therapists who are predicted to have a high alliance and/or therapeutic fit with the new patients.

In a third example, the plurality of health assessments 310 may be surgery-related health assessments that include information regarding the recovery health of patients, such as the temperature of the respective patient (which may indicate infection), how quickly a surgical wound is healing, or the patient's mental state in dealing with the physical trauma or surgery. Information from the health assessments 310 is then stored in Outcomes and Experiences data 246 and used to train one or more therapeutic referral model(s) 120 to match new surgical patients to surgeons who are predicted to have a high therapeutic fit with the new patients (e.g., the matched surgeon(s) may have a history of fast post-surgery recovery rates for patients undergoing the same surgical procedure and/or patients having a similar medical history or risk for complication as the new patient).

A sufficient number of health assessments 310 and a sufficient information (e.g., data) size in the Outcomes and Experience Data 246 is required for the therapeutic referral model(s) 120 to be trained. In some implementations, the health assessments 310 must include information for a plurality of different clinicians and each clinician must have treated at least 40 different patients for at least two treatment sessions each.

As more health assessments 310 are collected and information in the Outcomes and Experience Data 246 is updated, the therapeutic referral model(s) 120 may be periodically updated accordingly.

In some implementations, emergency room utilizations, hospital admissions, and/or medical co-morbidity rates may be used to test the therapeutic referral model(s) 120.

FIG. 3B provides an example of a health assessment 310 according to some implementations. As shown in FIG. 3B, the health assessment 310 may include patient information 340, such as patient name, date of birth (DOB), race/ethnicity, and medical history. The health assessment 310 may also include one or more patient preferences 342 (which may correspond to other user information or preferences 128) such as a clinician location (e.g., within 5 miles of the patient's home address, in the same county as the patient's home or workplace, or within 10 miles of a route between the patient's home and workplace), clinician age, gender (e.g., the same gender as the patient), or the clinician's area of expertise (e.g., family therapist, marriage counselor, ophthalmologist, or chiropractor). The health assessment 310 may also include other information 344, such as the urgency of the patient's medical needs. For example, “urgent” may mean “poses risk for immediate harm to self or others or needs immediate medical intervention such as surgery,” whereas “non-urgent” identifies minor issues, such as mild cold symptoms or a first degree burn. The other information 344 may also include compatibility scores (e.g., effectiveness scores) for different care approaches. For example, a first care approach for a psychiatric patient may include group therapy, a second care approach for the psychiatric patient may include individual therapy, and a third care approach may include medication (e.g., with a specific prescription, dose, and/or duration). Each care approach may include notes or a score on effectiveness of the approach. The health assessment 310 may also specify condition progress 346 of the patient. The condition progress 346 may include summaries after each treatment session. For each treatment session, the condition progress 346 may include a condition score (e.g., 1—mild, 10—severe), a condition change (e.g., a difference between the condition score of the most recent treatment session compared to the condition score of the previous treatment session), a condition progress (e.g., a calculated or expected trend of patient condition progression), clinician notes, and a patient self-assessment (which may include notes and/or numerical score(s)).

The health assessment 310 also includes a plurality of health measures, including proprietary measures such as Behavioral Health Index (referred to herein as a composite score), Therapeutic Alliance, and adherence to medications (e.g., psychiatric medications and course medications such as antibiotics). The Behavioral Health Index, or composite score, is a measurement of overall health (e.g., behavioral or mental health) and is calculated based on a patient's responses to a predetermined set of questions that cover multiple domains, such as well-being, depression, anxiety, and functioning level. Each of the domains has a respective weight in the calculation of the composite score. In some implementations, the composite score is determined based on a plurality of components. For example, a questionnaire may include six questions that correspond to the composite score, and each question of the six questions corresponds to a component of the composite score. In this example, the composite score may have 6 components, each corresponding to a question. Note that the questionnaire itself may include more than the 6 questions, as some questions may correspond to other measures or demographic information. In some implementations, one or more components of the composite score and/or the composite score be used to test the therapeutic referral model(s) 120. In some implementations, the therapeutic referral model(s) 120 are generated via a neural network or a random forest of decision trees that utilize the composite score (e.g., at least a component of the composite score, one or more components of the composite score, a portion or subset of components of the composite score, or all components of the composite score).

The Therapeutic Alliance is a measure of a bond level (or bond strength) between the patient and the clinician. Patients are asked to assess their Therapeutic Alliance via questions in the health assessment 310 after each treatment session, starting with the first post-treatment health assessment and continuing for the duration of the patient's care with the same clinician. An example of a question that measure Therapeutic Alliance is “Indicate whether or not you agree with the following statements: 1) In my last session, I felt heard, understood, and respected (agree/disagree), 2) In my last session, I understand and agree with how we are approaching my concerns.” Patient-reported outcomes, such as the Therapeutic Alliance, are useful in testing the validity of the therapeutic referral model(s) 120 as well as providing insight to symptomatology.

In some implementations, health measures also include one or more gold standard health tools, such as PHQ9 (a tool for measuring depression severity), GAD7 (a screening tool and symptom severity measure for the four most common anxiety disorders: generalized anxiety disorder, panic disorder, social phobia, and post-traumatic stress disorder (PTSD)), PCL-PC (a measure of PTSD developed by the Veteran's Administration for use in primary care settings), CSSR-S(Columbia Suicide Severity Rating Scale), and Alcohol Use Disorders Identification Test (AUDIT) (a measure of hazardous alcohol consumption). In some implementations, health measures may also include one or more diagnostic measures such as measures for substance abuse, drug abuse, bipolar disorder, depression, obsessive compulsive disorder (OCD), and/or psychosis. In some implementations, health measures may also include one or more predisposing factors such as treatment readiness, adherence to psychotherapy, coping skills, and/or subjective distress. In some implementations, health measures may also include one or more quality of life measures, such as a work adjustment measure, a familial/marital adjustment (e.g., domestic violence) measure, and/or a social adjustment measure.

In some implementations, an improvement or change is recorded for each measured health parameter, including proprietary health measures (such as Therapeutic Alliance) as well as gold standard measures (such as GAD7).

Thus, with each completed health assessment 310 (e.g., at the end or after a treatment session), the patient receives a new score for each of the health measures. The new scores provide the clinician and patient with an overall health score at a glance and can provide a quick comparison or observation of the patient's progress.

In some implementations, questionnaires and/or surveys associated with the health assessment 310 may be updated. For example, questionnaires or surveys used to collect information for a health assessment 310 may be updated periodically (e.g., once every month, once every year, when a gold standard health tool is updated, when a new gold standard health tool is released). The questionnaires or surveys may be updated to, for example, include new questions, exclude one or more questions, or change a weight of a domain. When the questionnaires or surveys are updated, the health assessment 310 will also be updated to include information corresponding to the updated questionnaires or surveys.

FIG. 3C provides an example of health progress according to some implementations. The plot 300 shown in FIG. 3C shows the composite score 350 (circles) of a patient in a patient-clinician pair. The plot 300 tracks the patient's progress over time (e.g., after each treatment session, after submission of a new filled-out questionnaire, or after receiving a new health assessment). In some implementations, the patient's composite score 350 is on a scale of 0 to 100. In some implementations, higher composite scores are indicative of more severe medical issues (e.g., mental health issues, physical health issues, overall health, or a health metric that corresponds to the medical issue that the patient is being treated for). In some implementations, the patient's composite score 350 corresponds to a percentile compared to other patients currently in the system (or in the system at the time of intake), or other patients currently receiving treatment (or receiving treatment at the time of intake). In some implementations, the patient's composite score 350 corresponds to a percentile compared to other patients with similar characteristics (e.g., the same diagnosis, the same gender, similar in age, the same treating clinician, the same treatment options, and/or similar medical histories). For example, a mental health patient with a composite score of 50 indicates that the patient is in the 50th percentile relative to mental health patients at intake. A patient with a composite score that is between 0 and 25 is considered to have “low” health issues, a patient with a composite score that is between 26 and 75 is considered to have a “moderate” health issues, and a patient with a composite score that is between 76 and 100 is considered to have a “severe” health issues. In the example shown in FIG. 3C, the patient appears to be improving, since the patient's composite score 350-6 at week 6 is at a lower percentile (˜24th percentile) compared to the patient's initial composite score 350-0 (˜50th percentile at week 0).

In some implementations, as shown in FIG. 3C, the plot 300 also shows an expected treatment response trend 352 of the patient's progress. The expected treatment response trend 352 is a prediction of how well the patient is expected to do over time provided that the patient continues treatment with the current clinician. In some implementations, the expected treatment response trend 352 is a prediction over a predefined or predetermined period of time, such as 10 weeks, 19 weeks, or a year after intake. The expected treatment response trend 352 is computed based on information received from the patient at intake and on normative data for other patients with similar characteristics at intake (e.g., similar age, gender, medical issues, severity, and medical history). In some implementations, the expected treatment response trend 352 is calculated using a regression analysis. In some implementations, the expected treatment response trend 352 is calculated using hierarchical linear modeling. The expected treatment response trend 352 illustrates a predicted (e.g., expected) change in the patient's composite score 350 over time. Thus, a comparison between a patient's composite score 350 and the expected treatment response trend 352 can be beneficial in determining the patient's rate of progress. For example, FIG. 3C shows that the composite score 350-6 of the patient at week 6 is below the expected treatment response trend 352, indicating that the patient is doing better than expected or predicted.

Additionally, the therapeutic referral model(s) 120 may layer on differential effects of baseline patient factors (e.g., the patient's overall physical and/or mental health, age, gender, and/or number of completed treatment sessions) and the effect of the treatment sessions (e.g., therapy provided by the counselor in a treatment session, nutritional advice provided in a treatment session, or chiropractic adjustments provided in a treatment session).

FIG. 3D illustrates a box plot 302 showing the effects of providing personalized referrals according to some implementations. Box plot 302 illustrates change in the composite score 350 for each patient 110 based on the percentage match of their treating clinician. As shown, a first group of patients that received treatment from a clinician that has a match of 97.5% or greater with the respective patient had a median change of −14.5 points in their composite score, representing an improvement in behavioral health index. Compared to the first group of patients, a second group of patients that received treatment from a clinician that has a match of 97.5% or lower with the respective patient had a median change of −10.5 points in their composite score. While the median change of both groups indicate an improvement in the health or condition of the patients, the first group of patients have a higher median change in composite score. The p-value of the difference in the median change in composite score between the first and second groups is 0.04, indicating that the difference in the median change in composite score between the two groups is statistically significant (indicating that the observed change is not likely to be caused by chance or randomness). As indicated by the oval region 304, when the match is at least 90%, the outcomes are better than clinician assignments with less of a match.

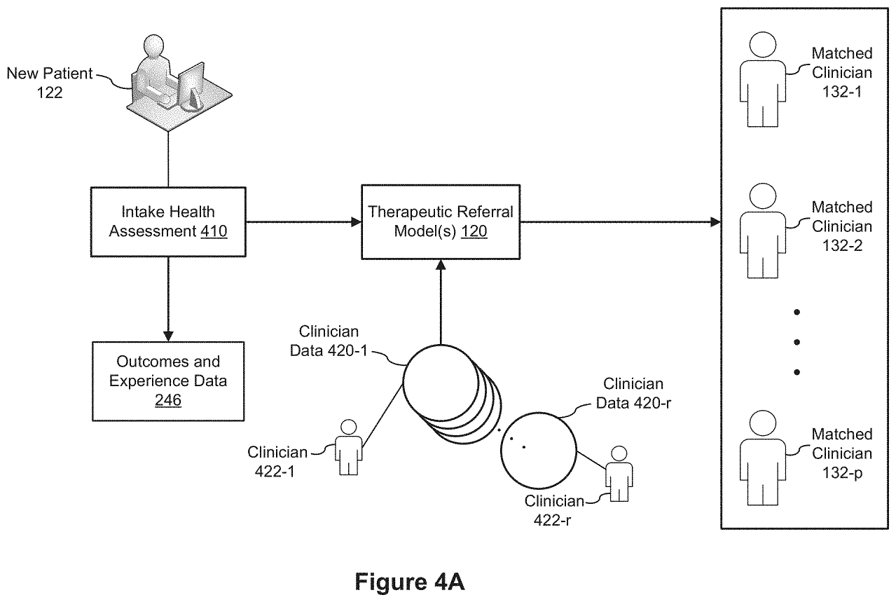

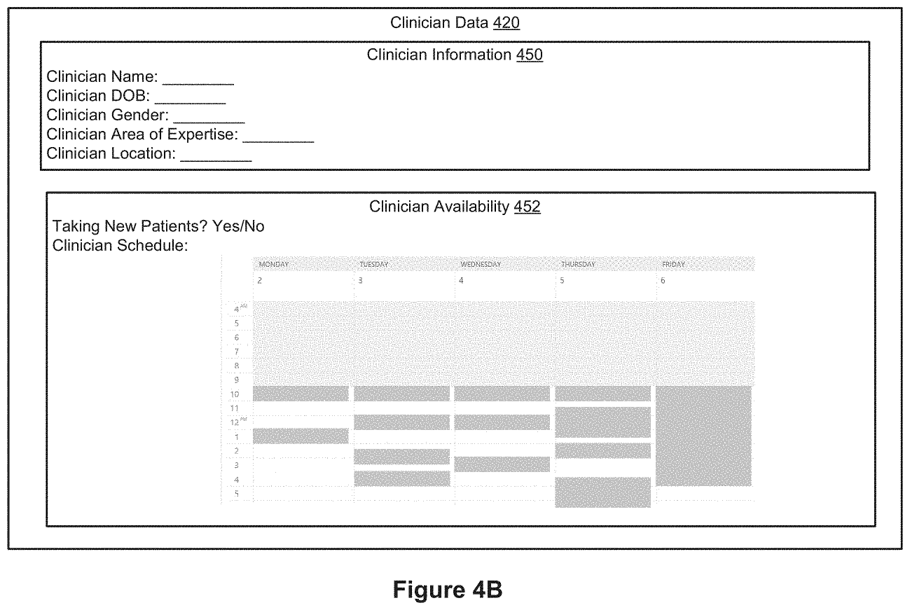

FIG. 4A illustrates using therapeutic referral model(s) 120 for providing healthcare referrals according to some implementations. When a new patient 122 is looking for a healthcare referral, the new patient 122 completes an intake health assessment 410. Information gathered by the intake health assessment 410 may include new patient information 124, a health assessment 126 (e.g., a behavioral health assessment, a mental health assessment, a physical health assessment, and/or a pre/post-surgical health assessment), and/or other user information or preferences 128, as described with respect to FIG. 1B. The intake health assessment 410 may also include questions regarding urgency, such as questions regarding mental state, amount of blood loss, or a likelihood or probability of harm to self or others. The intake health assessment 410 may also include a respective computed score for each health parameter (e.g., a Behavioral Health Index or an AUDIT value). For example, the intake health assessment 410 may include a composite score 350 (corresponding to an initial composite score 350-0 at week 0) based on the new patient's responses in the intake health assessment 410.

Information from the intake health assessment 410 is then included and stored as part of the Outcomes and Experience Data 246 and may be used in future updates and/or training of the therapeutic referral model(s) 120. The therapeutic referral model(s) 120 receive information from the intake health assessment 410 as well as clinician data 420 for a plurality of clinicians 422. Clinician data 420 for a given clinician may include clinician selection characteristics for the respective clinician. For example, first clinician data 420-1 for a first clinician 422-1 may include information such as the clinician's expertise, office location, gender, schedule, and whether or not the clinician is accepting new patients. The therapeutic referral model(s) 120 utilizes the information from the intake health assessment 410 and predicts the likely benefit that a candidate clinician would provide to the new patient 122.

The therapeutic referral model(s) 120 then generate a list of matched clinicians 132 that the therapeutic referral model(s) 120 predict to have a high likelihood of benefit to the patient. The generated list is personalized for the new patient 122 and the therapeutic referral model(s) 120 take into consideration a likelihood of patient-clinician alliance (e.g., compatibility) and therapeutic fit (e.g., the clinician's field of expertise overlaps with the patient's medical referral needs and the clinician has had success treating patients with the same disease and similar characteristics as the new patient 122).

In some implementations, the generated list of matched clinicians 132 is a ranked or prioritized list of clinicians. For example, the first matched clinician 132-1 would have a match score or likelihood of providing benefit to the new patient 122 that is higher than the second matched clinician 132-2. In some implementations, the new patient 122 is provided with a plurality of the matched clinicians 132. In some implementations, the new patient 122 is provided with all of the matched clinicians 132. In some implementations, the new patient 122 is provided with a subset of the matched clinicians 132. In some implementations, the new patient 122 is provided with one matched clinician at a time. For example, the first matched clinician 132-1 (e.g., the matched clinician with the highest match score) is presented to the user and in the case where the first matched clinician 132-1 is rejected (e.g., the office is too far away or the clinician has an incompatible schedule) the second matched clinician 132-2 is presented to the new patient 122 and so on until the new patient 122 is able to select and schedule an appointment with a matched clinician 132 from the generated list.

For example, a new patient 122 (a 57-year old male) may call a hotline or a call center for help. The hotline or call center employee may ask the new patient 122 questions from the questionnaire or survey in order to collect information as part of the intake health assessment 410. In some cases, the questionnaire or survey may be brief (e.g., 6 questions or less, 10 questions or less, 15 questions or less) in order to provide the new patient 122 with a fast response. The new patient's responses to the questions are input into the therapeutic referral model(s) 120, and a list of one or more matched clinicians 132 is provided. The model may be able to determine, based on the new patient's answers, that the new patient requires or is seeking a referral for drug-related counseling. The questions may appear on one or more questionnaires. The new patient 122 may provide, for example, demographic information such as age and gender, the new patient's medical history, substances and/or medication that the new patient 122 is currently taking, as well as any clinician preferences such as a location-based preference (e.g., within 5 miles of the patient's zip code) or a gender preferences (e.g., the same gender as the new patient 122, or “male”). In some instances, a prospective patient answers the questions on paper (e.g., if visiting an advice nurse in person). In some instances, the prospective patient answers the questions online (e.g., using a web-based application or website). In some instances, the prospective patient provides answers to the questions verbally (e.g., speaking to a call center representative, who inputs the answers into an electronic application or database).

The therapeutic referral model(s) 120 utilize information provided by the new patient 122 via the intake health assessment 410 and provides the new patient 122 with one or more matched clinicians 132. For example, the first matched clinician 132-1 may be a 98% match to the new patient 122. The first matched clinician 132-1 may have a high predicted likelihood of benefit to the new patient 122 based on the clinician's experience and high success rate of treating men aged between 50-60 years for the same substance abuse problem as identified by the new patient 122. Additionally, the first matched clinician 132-1 may also meet a majority of the new patient's preferences, such as scheduling compatibility, gender preference, and/or location preference. Compared to the first matched clinician 132-1, the lower-ranked clinicians, are predicted by the therapeutic model(s) to be a poorer match or fit to the new patient 122. For example, the second matched clinician 132-2 may be a 97.8% fit to the new patient 122, lower than the first matched clinician 132-1 (with a match of 98%) due to a location of the second matched clinician's office not fitting within the user's preferences. Alternatively, the second matched clinician 132-2 may have a lower predicted match compared to the first matched clinician 132-1 due to the second matched clinician 132-2 having a high success rate of treating men aged between 50-60 years but no history of treating men aged 57 years, whereas the first matched clinician 132-1 has treated at least 5 men aged 57 years with a high success rate.

In a second example, a new patient 122 (15 year old female) who needs surgery for a broken arm may fill out one or more questionnaires as part of the intake health assessment 410. The new patient 122 may provide, for example, demographic information such as age and gender, the new patient's medical history, as well as any clinician preferences such as a scheduling-based preference (e.g., as soon as possible). The therapeutic referral model(s) 120 utilize information provided by the new patient 122 via the intake health assessment 410 is and provides the new patient 122 with one or more matched clinicians 132. For example, the first matched clinician 132-1 may be a 99.5% match to the new patient 122. The first matched clinician 132-1 may have a high predicted likelihood of benefit to the new patient 122 based on the clinician's extensive experience in treating multiple fractures and fast post-surgery patient recovery rate (e.g., 95% of patients regain full strength within 12 weeks post-surgery). However, the first matched clinician 132-1 may currently be on vacation and will not be back until next week. The second matched clinician 132-2 may be a 99.3% fit to the new patient 122, lower than the first matched clinician 132-1 (with a match of 99.5%) due to a slightly slower post-surgery patient recovery rate (e.g., 93% of patients regain full strength within 15 weeks post-surgery).

Once an appointment is made, the new patient 122 attends the appointment for the first treatment session with the selected clinician from the list of matched clinicians 132. In some implementations, at the appointment, the new patient 122 fills out one or more questionnaires that correspond to a first or an initial health assessment that will be the first health assessment of a set of health assessments that correspond to the specific patient-clinician pair. The information in this initial health assessment may be used to track the progress of this patient as he/she works with this specific clinician. An example of the tracked progress of a patient in a patient-clinician pair is described above with respect to FIG. 3C. In this example, the initial health assessment may correspond to the initial composite score 350-0 at week 0.

FIG. 4B is an example of clinician data 420 according to some implementations. The clinician data 420 includes clinician information 450 such as demographic information and practice information. Examples of demographic information include date of birth (DOB), gender, race, ethnicity, languages spoken during treatment sessions (e.g., therapy sessions). Examples of practice information include type of degree (e.g., PhD, PsyD, LCSW, MD), years conducting treatment, field of expertise or specialty (e.g., addiction, anger, anxiety, bipolar disorder, borderline personality disorder, eating disorder, domestic violence, gender identity, relationship/marital issues, self-harm, trauma, sexuality issues, PTSD, suicidal ideation, and/or chronic illness), and/or types of therapy practiced (e.g., cognitive behavioral therapy (CBT), acceptance and commitment therapy (ABT), and/or faith-based therapy). The clinician data 420 may also include other information such as office location (e.g., address, zip code, whether or not the office is accessible by public transportation, is there available parking, is the office handicapped accessible), the clinician's availability 452 (e.g., the clinician's schedule and whether or not the clinician is currently accepting new patients), additional fees (e.g., new patient fee, late fees), pet policy (are pets allowed on premises/during treatment sessions), whether or not the clinician conducts home calls/visits or over the phone or web-conference sessions, whether or not the clinician is a Medicare provider, and/or what insurance plans the clinician accepts. The clinician data 420 may also include identifying information, such as the clinician's medical license number or certification number for a particular treatment method.

In some implementations, the clinician data 420 is acquired via an on-boarding questionnaire (e.g., survey), which is completed by a new or potential clinician at the on-boarding stage. The onboarding questionnaire may include any combination of open text answers, rating questions (e.g., on a scale of 1-10, rate your level of experience in treating patients with eating disorders), dichotomous questions (e.g., Yes/No questions), and checkbox questions (e.g., check all the areas in the list below that you specialize in or treat)

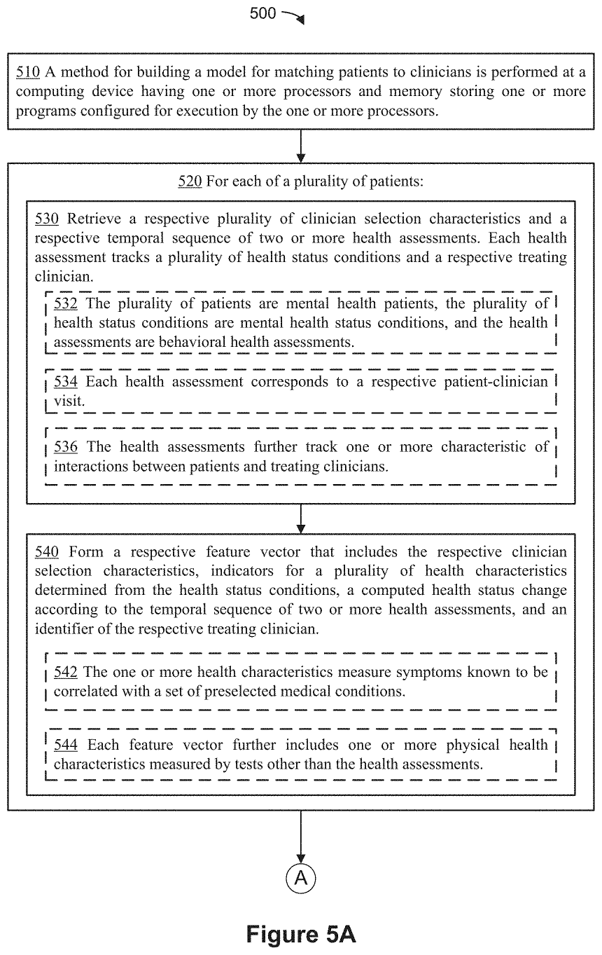

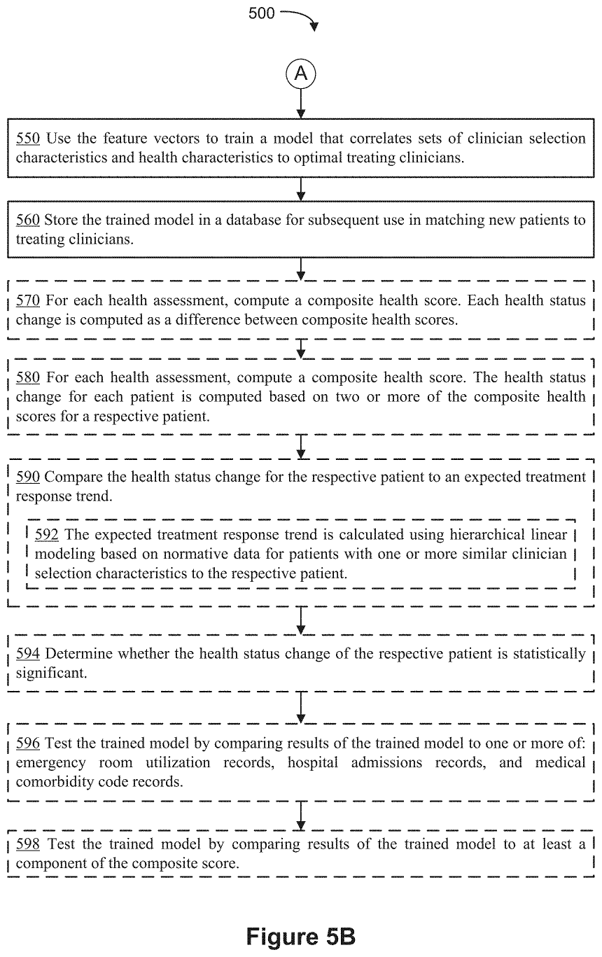

FIGS. 5A and 5B provide a flow diagram of a method 500 for building therapeutic referral model(s) 120 for matching patients to clinicians according to some implementations. The steps of the method 500 may be performed by a computer system, corresponding to a computer device 200 or a server 250. In some implementations, the computer includes one or more processors and memory. FIGS. 5A and 5B correspond to instructions stored in computer memory or a computer-readable storage medium (e.g., the memory 206 of the computing device 200). The memory stores (510) one or more programs configured for execution by the one or more processors. For example, the operations of the method 500 are performed, at least in part, by a health assessment module 236.

In accordance with some implementations, a computer system, computing device 200, or a server 250 performs (520) a series of operations for a plurality of patients 110. The system retrieves (530) a respective plurality of clinician selection characteristics (e.g., other user information of preferences 128 corresponding to information stored as part of clinician data 420) and a respective temporal sequence of two or more health assessments 310. Each health assessment 310 tracks a plurality of health status conditions and a respective treating clinician 112. For example, as shown in FIG. 3A, the computer system receives a plurality of health assessments 310 and each health assessment 310 corresponds to a patient-clinician pair 114-1. The computer then forms (540) a respective feature vector that includes the respective clinician selection characteristics, indicators for a plurality of health characteristics, a computed health status change, and an identifier of the respective treating clinician (e.g., a medical license number, name). The indicators for the plurality of health characteristics are determined (540) from the health status conditions and the computed health status change is determined (540) according to the temporal sequence of two or more health assessments. The computer then uses (550) the feature vectors to train a model 120 that correlates a set of clinician selection characteristics and health characteristics to optimal treating clinicians and stores (560) the trained model in a database for subsequent use in matching new patients to treating clinicians.

In some implementations, the plurality of patients 110 are (532) mental health patients, the plurality of health status conditions are mental health status conditions, and the health assessments 310 are behavioral health assessments.

In some implementations, each health assessment 310 corresponds (534) to a respective patient-clinician visit (e.g., a fifth treatment session for a patient 110-1 being treated by a clinician 112-1).

In some implementations, the health assessments 310 further track (536) one or more characteristic of interactions between patients and treating clinicians. For example, the health assessments 310 may include, in addition to a health status condition of the patient 110, a patient evaluation of the treating clinician (e.g., “ 9/10—I like working with this doctor. She listens to my questions and provides responses that address my concerns.”)

In some implementations, the one or more health characteristics measure (542) symptoms known to be correlated with a set of preselected medical conditions. For example, the one or more health characteristics may include patient temperature, redness at surgical site, swelling of the surgical wound—each of which is correlated with infection. In another example, the one or more health characteristics may include mood swings, insomnia, and a sudden change (increase or decrease) in appetite—each of which is correlated with one or more behavioral or mental disorders.

In some implementations, each feature vector further includes (544) one or more physical health characteristics measured by tests other than the health assessments. For example, a feature may include information such as whether or not the patient has had surgery before, any medications that the patient is allergic to or is currently taking, or any past treatments that the patient may have undergone and found beneficial/not beneficial.

In some implementations, for each health assessment 310, the computer system computes (570) a composite health score 350 and each health status change is computed (570) as a difference between composite health scores 350.

In some implementations, for each health assessment 310, the computer computes (580) a composite health score 350. The health status change for each patient is computed (580) based on two or more of the composite health scores for a respective patient. For example, FIG. 3C shows a series of composite health scores 350 for a respective patient. In this example, a health status change for the respective patient between week 0 and week 5 is approximately −25. In some implementations, the current health status change is calculated based on the two most recent composite scores (e.g., the health status change for week 6 would be the difference between the composite score of week 6 and week 5). In some implementations, a current health status change may be calculated based on the difference between the most recent composite score and the composite score 350-0 at intake (e.g., the composite score 350-0 at week 0).

In some implementations, the computer compares (590) the health status change for the respective patient to an expected treatment response trend 352. For example, the computer may calculate the difference between the health status change for the respective patient to an expected treatment response trend 352 and provide a comparison in the form of a numerical value or a data visualization or plot.

In some implementations, the expected treatment response trend 352 is calculated (592) using hierarchical linear modeling based on normative data for patients with one or more clinician selection characteristics similar to those of the respective patient.

In some implementations, the computer determines (594) whether the health status change of the respective patient is statistically significant.

In some implementations, the computer tests (596) the trained model 120 by comparing results of the trained model to one or more of: emergency room utilization records, hospital admissions records, and medical comorbidity code records.

In some implementations, the computer tests (598) the trained model 120 by comparing results of the trained model to at least a component of the composite score.

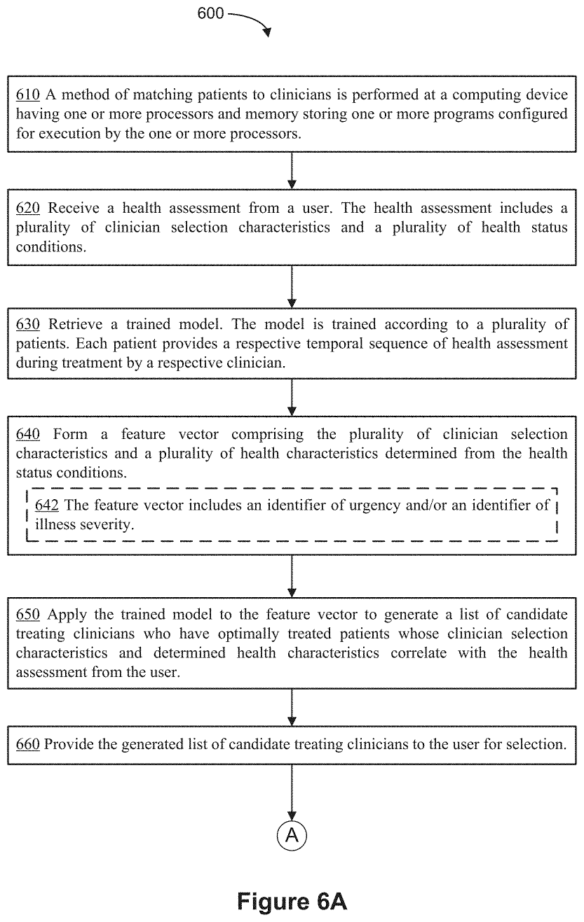

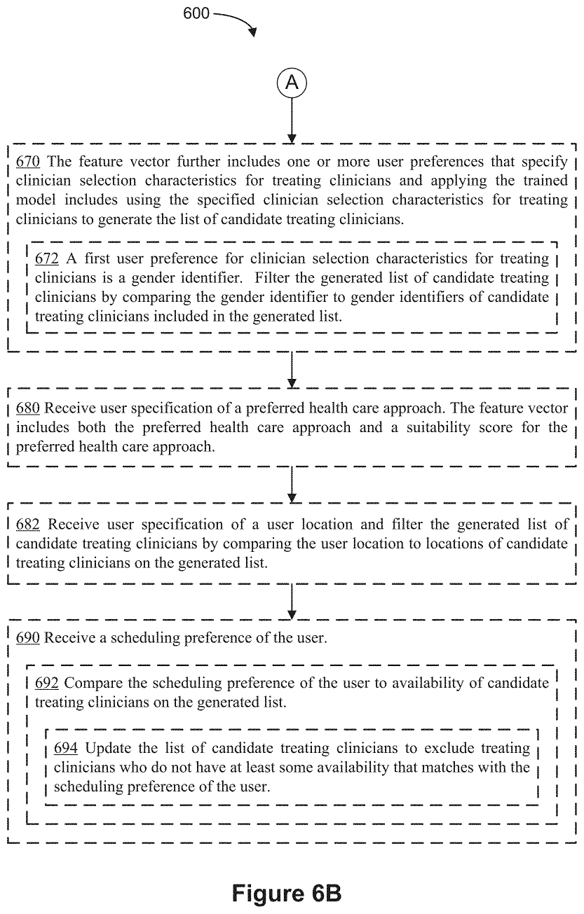

FIGS. 6A and 6B provide a flow diagram of a method 600 for matching patients to clinicians according to some implementations. The steps of the method 600 may be performed by a computer system, corresponding to a computer device 200 or a server 250. In some implementations, the computer includes one or more processors and memory. FIGS. 6A and 6B correspond to instructions stored in a computer memory or computer-readable storage medium (e.g., the memory 206 of the computing device 200). The memory stores one or more programs configured for execution by the one or more processors. For example, the operations of the method 600 are performed (610), at least in part, by a health assessment module 236.

In accordance with some implementations, a computer system or computing device 200 receives (620) a health assessment 410 from a user (e.g., a new patient 122). The health assessment 410 includes (620) a plurality of clinician selection characteristics and a plurality of health status conditions. The computer then retrieves (630) a trained model 120. The trained model 120 has been trained (630) according to a plurality of patients. Each patient provided (630) a respective temporal sequence of health assessments 310 during treatment by a respective clinician 112, described above with respect to FIGS. 3A and 5A-5B. The computer then forms (640) a feature vector comprising the plurality of clinician selection characteristics and a plurality of health characteristics determined from the health status conditions and applies (650) the trained model 120 to the feature vector to generate a list 130 of candidate treating clinicians 132 who have optimally treated patients whose clinician selection characteristics and determined health characteristics correlate (e.g., are similar to or overlap at least partially) with the health assessment from the user 122. The computer then provides (660) the generated list 130 of candidate treating clinicians 132 to the user 122 for selection.

In some implementations, the feature vector includes (642) an identifier of urgency and/or an identifier or illness severity.

In some implementations, the feature vector further includes (670) one or more user preferences 128 that specify clinician selection characteristics for treating clinicians 422. Applying the trained model 120 includes (670) using the specified clinician selection characteristics for treating clinicians 422 to generate the list 130 of candidate treating clinicians 132.

In some implementations, a first user preference for clinician selection characteristics for treating clinicians 422 is (672) a gender identifier. The computer filters (672) the generated list 130 of candidate treating clinicians 132 by comparing the gender identifier to gender identifiers of candidate treating clinicians 422 included in the generated list 130. For example, the computer may compare a gender identifier in clinician data 420-1 for a first clinician 422-1 to a gender identifier provided by the new patient 122 as a user preference.

In some implementations, the computer receives (680) user specification of a preferred health care approach. The feature vector includes (680) both the preferred health care approach and a suitability score for the preferred health care approach. For example, the new patient 122 may indicate that he/she prefers individual therapy instead of group therapy. The feature vector may also include a suitability score for individual therapy for this patient 122. The score may be based in part on the indicated user preference for individual therapy and/or records indicating the level of benefit that individual therapy has provided to other patients with similar characteristics (e.g., medical concerns, medical history, demographic information) to the new patient 122.

In some implementations, the computer receive (682) user specification of a user location and filters the generated list 130 of candidate treating clinicians 132 by comparing the user location to locations of candidate treating clinicians 132 on the generated list 130.

In some implementations, the computer receives (690) a scheduling preference of the user, compares (692) the scheduling preference of the user to availability of candidate treating clinicians 132 on the generated list 130, and updates (694) the list 130 of candidate treating clinicians 132 to exclude treating clinicians who do not have at least some availability that matches the scheduling preference of the user.

In some implementations, the computer is a wearable device. For example, the computer may be a wearable display that includes one or more input means (such as a microphone, joystick, buttons, touchpad, mouse, or keyboard), a head-mounted display device (such as a virtual-reality display device, an augmented-reality display device, smart glasses or smart goggles), or a smart accessory (such as a smart watch or fitness tracker with one or more input means).

The terminology used in the description of the invention herein is for the purpose of describing particular implementations only and is not intended to be limiting of the invention. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The foregoing description, for purpose of explanation, has been described with reference to specific implementations. However, the illustrative discussions above are not intended to be exhaustive or to limit the invention to the precise forms disclosed. Many modifications and variations are possible in view of the above teachings. The implementations were chosen and described in order to best explain the principles of the invention and its practical applications, to thereby enable others skilled in the art to best utilize the invention and various implementations with various modifications as are suited to the particular use contemplated.

What is claimed is:

1. A method for building a model for matching patients to clinicians, performed at a computing device having one or more processors and memory storing one or more programs configured for execution by the one or more processors:
for each of a plurality of patients:
retrieving a respective plurality of clinician selection characteristics and a respective temporal sequence of two or more health assessments, each health assessment tracking a plurality of health status conditions and a respective treating clinician;
forming a respective feature vector comprising the respective clinician selection characteristics, indicators for a plurality of health characteristics determined from the health status conditions, a computed health status change according to the temporal sequence of two or more health assessments, and an identifier of the respective treating clinician;
using the feature vectors to train a model that correlates sets of clinician selection characteristics and health characteristics to optimal treating clinicians; and
storing the trained model in a database for subsequent use in matching new patients to treating clinicians.

2. The method of claim 1, wherein:
the plurality of patients are mental health patients;
the plurality of health status conditions are mental health status conditions; and
the health assessments are behavioral health assessments.

3. The method of claim 2, wherein each feature vector further comprises one or more physical health characteristics measured by tests other than the health assessments.

4. The method of claim 1, wherein each health assessment corresponds to a respective patient-clinician visit.

5. The method of claim 1, further comprising:
for each of the health assessments, computing a respective composite health score, wherein each health status change is computed as a difference between composite health scores.

6. The method of claim 1, further comprising, for each of the health assessments, computing a respective composite health score, wherein:
the health status change for each patient is computed based on two or more of the composite health scores for a respective patient.

7. The method of claim 6, further comprising:
comparing the health status change for the respective patient to an expected treatment response trend.

8. The method of claim 7, wherein the expected treatment response trend is calculated using hierarchical linear modeling based on normative data for patients having one or more clinician selection characteristics that match the clinician selection characteristics of the respective patient.

9. The method of claim 6, further comprising:
determining whether the health status change of the respective patient is statistically significant.

10. The method of claim 6, further comprising:
testing the trained model by comparing results of the trained model to at least a component of the composite score.

11. The method of claim 1, wherein the health assessments further track one or more characteristics of interactions between patients and treating clinicians.

12. The method of claim 11, wherein the one or more characteristics measure symptoms known to be correlated with a set of preselected medical conditions.

13. The method of claim 1, further comprising:
testing the trained model by comparing results of the trained model to one or more of: emergency room utilization records, hospital admissions records, and medical comorbidity code records.

14. A computer system for matching patients to clinicians, comprising:
one or more processors;
memory; and
one or more programs stored in the memory and configured for execution by the one or more processors, the one or more programs comprising instructions for:
for each of a plurality of patients:
retrieving a respective plurality of clinician selection characteristics and a respective temporal sequence of two or more health assessments, each health assessment tracking a plurality of health status conditions and a respective treating clinician;
forming a respective feature vector comprising the respective clinician selection characteristics, indicators for a plurality of health characteristics determined from the health status conditions, a computed health status change according to the temporal sequence of two or more health assessments, and an identifier of the respective treating clinician;
using the feature vectors to train a model that correlates sets of clinician selection characteristics and health characteristics to optimal treating clinicians; and
storing the trained model in a database for subsequent use in matching new patients to treating clinicians.

15. The computer system of claim 14, wherein:
the plurality of patients are mental health patients;
the plurality of health status conditions are mental health status conditions; and
the health assessments are behavioral health assessments.

16. The computer system of claim 14, wherein each health assessment corresponds to a respective patient-clinician visit.

17. The computer system of claim 14, further comprising:
for each of the health assessments, computing a respective composite health score, wherein the health status change for each patient is computed based on two or more of the composite health scores for a respective patient.

18. A non-transitory computer readable storage medium storing one or more programs configured for execution by a computer system having one or more processors, memory, and a display, the one or more programs comprising instructions for:
for each of a plurality of patients:
retrieving a respective plurality of clinician selection characteristics and a respective temporal sequence of two or more health assessments, each health assessment tracking a plurality of health status conditions and a respective treating clinician;
forming a respective feature vector comprising the respective clinician selection characteristics, indicators for a plurality of health characteristics determined from the health status conditions, a computed health status change according to the temporal sequence of two or more health assessments, and an identifier of the respective treating clinician;
using the feature vectors to train a model that correlates sets of clinician selection characteristics and health characteristics to optimal treating clinicians; and
storing the trained model in a database for subsequent use in matching new patients to treating clinicians.

19. The non-transitory computer readable storage medium of claim 18, wherein:
the plurality of patients are mental health patients;
the plurality of health status conditions are mental health status conditions; and
the health assessments are behavioral health assessments.

20. The non-transitory computer readable storage medium of claim 18, further comprising, for each of the health assessments, computing a respective composite health score, wherein:
the health status change for each patient is computed based on two or more of the composite health scores for a respective patient.
